(19) United States
(12) Patent Application Publication
(10) Pub. No.: US 2017/0164882 A1
(43) Pub. Date: Jun. 15, 2017

(54) (SSCD) DEVICE - SAFETY SYRINGE COLLECTION DEVICE

(71) Applicant: Uneeka Canty, Lake Wales, FL (US)

(72) Inventor: Uneeka Canty, Lake Wales, FL (US)

(21) Appl. No.: 14/963,417

(22) Filed: Dec. 9, 2015

(51) Int. Cl. A61B 5/15

(52) U.S. Cl. CPC A61B 5/150236; A61B 5/150244; A61B 5/150519; A61B 5/150251

(57) ABSTRACT

A Safety Syringe Collection Device (SSCD) Device used for collecting specimen (fluids) from any line device with one process and one device. The SSCD Device invention has a housing barrel to abstract fluids, second barrel is a hollow barrel with an attached rubber stopper with small openings/holes at both ends to allow free flow through it, and hold fluids until transferred in a collection container(s) at distal end of the second barrel where the sharp object and rubber cover that the container(s) are connected to for collection of fluids. The syringe versatile mode of transferring hazardous fluids mean the operator and patient's safety. The collection process is preferably synchronized with the syringe to collect and transfer fluid(s) through the SSCD Device which will have as easier mode for collection, transfer, cost effective, safety, and use.

### CROSS-REFERENCES TO RELATED APPLICATION(S)

Not Applicable.

### STATEMENT REGARDING FEDERALLY SPONSORED RESEARCH OR DEVELOPMENT

Not Applicable.

### MICROFICHE APPENDIX

Not Applicable.

### BACKGROUND OF THE INVENTION

1. Field of Invention

The present invention relates to the medical field and a medical device.

2. Background of the Invention

Medical personnel that are licensed to access a line or port of any kind to give medication or collect specimen (fluids). The conventional syringe collection process through a tube. Accessing the line or port of any kind is done to give medication or most likely for collecting specimen (fluids) that are being tested for whatever reason(s) as well as in many different collection container(s).

Typically in collecting one or multiple specimens or (fluid) can pose difficulty in the process. An example of this difficulty is the traditional collection process was done by opening a syringe, connecting to any distal end of a line, being pulled to abstract the fluids, the syringe has to be dis-attached, it has to be connected to a needle after being uncapped, once collected into the syringe its then recapped again for securement of specimen and needle stick, the syringe needle has to be uncapped again to transfer specimen into a collection container, the needle needs to be recapped for safety of needle sticks for patient and healthcare worker before discarding into bio-hazard container after transferring specimen into the collection container(s). However, a syringe supposed to operate on the principles of simple collection but causes needle sticks, spillage and too many steps for collection, transfer, use and disposal.

The patient has to have specimen collected to let physicians know if medications or treatments are working. Although in this situation it can be useful but it still can be problematic. Once you collect the specimen in the syringe and with different test that have to be ran, the syringe holds 10 ml of fluids or a little more. If more is needed, then the whole process will need to be repeated to collect more fluids.

The existing art describes the mode of transportation of fluids for collection through barrels designed to enter directly into collection container from the barrels. It has an easy disposal with collection attachments already attached to the device. If more specimen is needed you can just attach to bottom and collect. The SSCD device can be left attached to line without injury until all specimen needed is collected especially in emergency settings.

Previously, this was done by a single syringe. And as such, the SSCD device allows multifunction collection to maintain safety and non-contaminated fluids. In an attempt to produce a multifunctional device this system, while providing a more consistent mode of transportation as well as safer collection process. The traditional process lacks the means of safer and easier mode of transportation with less steps to operate.

SSCD Device is an object of this invention to create a syringe to collect multiple specimens (fluids) for testing, etc.

The SSCD Device is another object of this invention to create a syringe without spillage. For example, once the SSCD Device is connected, it doesn't have to be disconnected from the line for transfer or collecting. Spillage happens when the traditional syringe is disconnected for needle attachment before collection.

The SSCD Device is another object of this invention to create a syringe to prevent needle sticks. For example, most of the needle stick incidents come from recapping or trying to aim needle in the collection container. And this can happen before or after needle is contaminated. SSCD Device prevent most needle sticks because needle is housed in the inside of the second barrel which protect the needle as well as the user.

The SSCD Device is another object of this invention to create a syringe with easy collection of specimens (fluids) also multiple collection. For example, with the traditional process a needle has to be connected to syringe and then aimed into container. Whereas the SSCD Device doesn't need to be disconnected from the host to collect specimen because needle is made in the second barrel where you can just push on any collection container and collect specimen. Because the specimen can flow freely through the housing barrel and then into the second barrel, there is no need to reconnect a new device to obtain more specimen if needed, just continue to change collection container until you're done.

The SSCD Device Is another object of this invention to create a syringe with less steps in the whole process for example, the SSCD device you attach once, collect multi-specimen through second barrel then disconnect and discard. There is no need for recapping or reconnecting other components for one process and to deal with contaminated specimen. The SSCD Device can also stay connected in an emergency setting without injury to healthcare workers or patient if necessary.

The SSCD Device is another object of this invention to create a syringe that is cost effective. For example, the traditional process you have to purchase the syringe and needles separately. You would have to have more than one syringe to collect a large amount of specimen. The SSCD Device can collect multi-specimen with one multi-functional device that will save on cost. The traditional syringe use if you don't have one without the other the procedure can't be done.

There are some known devices for collection but the most common one is pulling fluids from any type of line from a patient or (source) where the syringe has to be connected. After connection and fluids have been collected it has to be disconnected. Once disconnected the operator(s) have to connect a separate needle attachment, then pull off needle covering. Re-attach the needle cover until it can be transferred. Once it's ready to be transferred you have to uncap the needle and place the needle part into the transferable container(s). The components are sold separately as well as having to be assembled before use and after. The conventional way has too many incidents and problems that need to be corrected. As with myself and so many other healthcare workers incident(s) are needle sticks, contaminated specimen contact, contaminated specimens, clotted specimens and spillage because of leaking. The new collection syringes have the same process except at the end you can pull down on the barrel and needle sit inside of it. So you don't have to disconnect the needle at the end. Now with the new syringe there are still just as many needle sticks because the operator still has to transfer specimen after it's collected in the syringe as well as if more specimen is needed the process will have to be done again to obtain more specimen. You still have to purchase the needles separately as well as connect before you can use the device. Needle is still exposed before and after use, which means you still have a high rate of needle stick exposure for the patient and healthcare workers. The SSCD Device you have no direct contact with the needle to prevent needle sticks.

Presently there is no solution for the present process to make it more effective, shorter steps and cost efficient and most of all safer for healthcare workers.

### BRIEF SUMMARY OF ITS BASIC FUNCTION

The head of the collection device connects directly to the distal end of any line open host for direct attachment. Which helps with less contact with the body fluids and spillage. The hollow tube allows specimen (fluid) to fill up in it as well as the base which allows other containers to connect, specimen(s) go directly in the container to prevent needle sticks, non-contaminated specimens and compromised specimen for testing.

The head of the device attach directly on by screwing on as stated in claim 1. The hollow housing barrel tube can fill with specimen (fluid) with a gentle pull to begin flow in the tube. The fluid flows through an opening at the top of the second barrel. The fluid is stored (set) in the second barrel in the tube until a bottom container is placed at the base for collection. The specimen (Fluids) flow through a small attachment at the base of the second barrel where fluids set in the sharp object until containers are pushed on to activate collection.

Important aspects of the present invention is no contact with fluids, no spillage, no needle sticks, no compromised specimens for testing, no assembly and no purchase of different units to do one job.

In emergency settings such as hospital emergency room(s) it can stay attached until fluids are collected or detached and still have all the specimen (fluids) you need. You're out of the other healthcare workers' space and able to still do your collection without compromised specimen(s).

The Important Aspects of the (SSCD) Device

The head of the device attach directly on by screwing on with two or more twist. The hollow housing barrel tube can fill with specimen (fluid) with a gentle pull to begin flow in the tube. The fluid flows through an opening at the top of the second barrel. The fluid is stored (set) in the second barrel in the tube until a bottom container is placed at the base for collection. The specimen (Fluids) flow through a small attachment at the base of the second barrel where fluids set in the sharp object until containers are pushed on to activate collection. Important aspects of the present invention are no contact with fluids, no spillage, no needle sticks, no compromised specimens for testing, no assembly and no purchase of different units to do one job. In an emergency setting such as hospital emergency room(s) it can stay attached until fluids are collected or detached and still have all the specimen (fluids) you need. You're out of the other healthcare workers' space and able to still do your collection without compromised specimen(s).

### BRIEF DESCRIPTION OF SEVERAL VIEWS OF THE DRAWING

The drawings presently illustrate the preferred embodiments of the invention as well as the description that follows, to explain the principles of the invention.

FIGS. 1, 2 and 3 depict embodiments of the invention with a separate views of the assembly of the hand held device.

FIG. 1 is a perspective view, which depict the present invention as a whole unit as it would come together in packaging. A preview of fluid in the barrel to show how it would work as well as its other components.

FIG. 2 is an exploded perspective view, which depict the second barrel separate from the housing barrel. The view of the second barrel and all its components to carry out its functions.

FIG. 3 is an exploded perspective view, which depict the housing barrel and all of its components separate from the second barrel.

FIG. 4 is an exploded perspective view of FIG. 2, which depict the second barrel separate from the housing barrel. The view of the second barrel and all its components to carry out its functions.

### REFERENCE NUMBERS IN THE DRAWINGS

1. Nozzle Head
2. Screw Attachment
3. Fluid in Housing Barrel
4. Top Small opening/hole
5. Bottom Small opening/hole
6. Second Barrel
7. Small Attachment
8. Left Finger Bridge
9. Right Finger Bridge
10. Bottom Base of Barrel
11. Rubber Covering
12. Sharp Object
13. Lower Left Finger Bridge
14. Lower Right Finger Bridge
15. Rubber Stopper
16. Top Surface of Barrel
17. Opening/Second Barrel
18. Opening/Housing Barrel
19. Barrel Opening to Collection Site
20. Hollow inside of the Housing Barrel
21. Plastic attachment is the same shape and form of the rubber stopper.
22. The measuring scale for fluids.
23. The housing barrel.

### DETAILED DESCRIPTION OF THE INVENTION

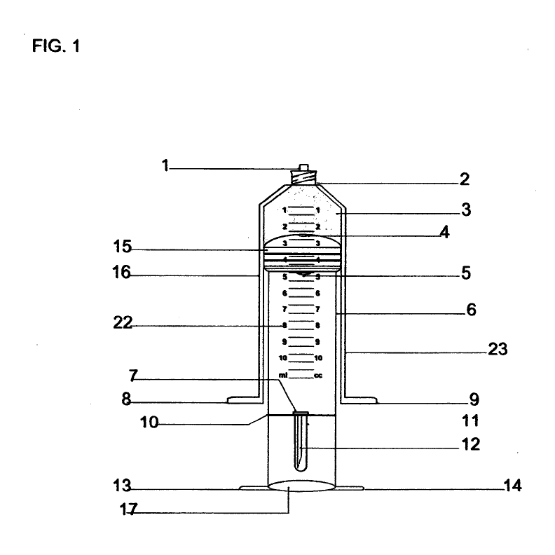

FIG. 1 shows a perspective view of the present invention in the assembled state. The top of the SSCD Device 1. It has a circular tip with a small opening to allow an open free flow. The top can be removed, not limited to any object or nothing at all but an open to allow free flow. 2 is a circular piece that is screwed onto other attachments (devices) to allow the device and any line to be connected without spillage. 3 is an example of any fluids, body or etc. Housing the hollow space of the housing barrel after device is in progress. 4 is an opening threw the top of the rubber stopper were fluids can pass through to the opposite side. 5 is a bottom opening to continue the free flow that is made on the rubber stopper. 6 is a clear plastic barrel with a hollow inside that is attached to the bottom of the rubber stopper an enclosed bottom with small opening. 7 is a circular object that is affixed to a small hole in the base of the barrel. 8 & 9 are finger bridges that are attached to the left and right base of the barrel for grip to push or pull and is not limited to being produced without the finger bridges. 10 is the plastic base closure of the barrel with a small opening in the center for 7, 11, 12 attachments and not limited to any other attachments to achieve the same goal. 11 is a rubber cover to protect the sharp object and free flow of fluids. 12 is a sharp object inside of 11 that allows fluids to flow and other attachments can connect to collect the fluids. 13 & 14 are finger bridges that allow control over the device and attachments. 15 is the rubber stopper to hold fluids and pressure when activated. 16 is the top of the second barrel flat base. 17 is the bottom opening for second barrel. 18 is the opening of the housing barrel were the second barrel is placed 19 is a sectioned part of the second barrel where 11 & 12 reside in the interior of the barrel.

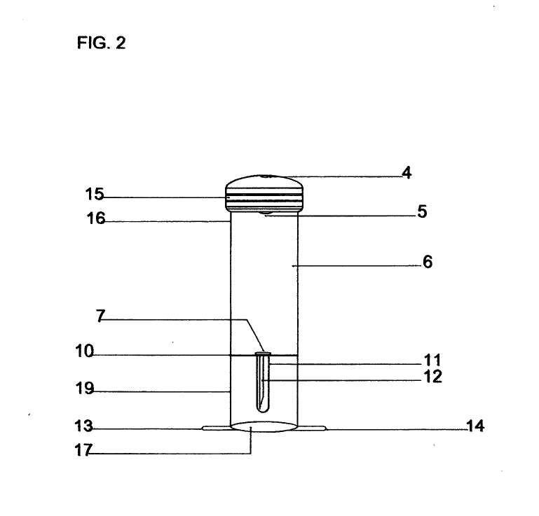

FIG. 2 is an exploded perspective view, showing the second barrel separate from the housing barrel. 4 is the small opening/hole on the top of the rubber stopper. 5 is the small opening/hole at the bottom of the rubber stopper which is attached to 16 is the surface enclosed second barrel with a small opening/hole in the center of it. 15 is the rubber stopper with to small openings/hole in the top and bottom of it and not limited to other attachments or none at all to accomplish the same goal. 6 is a clear plastic barrel with a hollow inside that is attached to the bottom of the rubber stopper an enclosed bottom with small opening. (16) The flat surface with a small hole/opening the sit in the center of it. 10 is the base of the hollow barrel with small opening/hole. 7 is a small attachment feature. Ills a rubber cover to protect the sharp object and free flow of fluids. 12 is a sharp object inside of 11 that allows fluids to flow free. 19 is a sectioned part of the second barrel where 11 & 12 reside for attachment container(s) for collecting fluids. 13 & 14 are finger bridges that allow control over the device and attachments. 17 is the bottom opening of the second barrel.

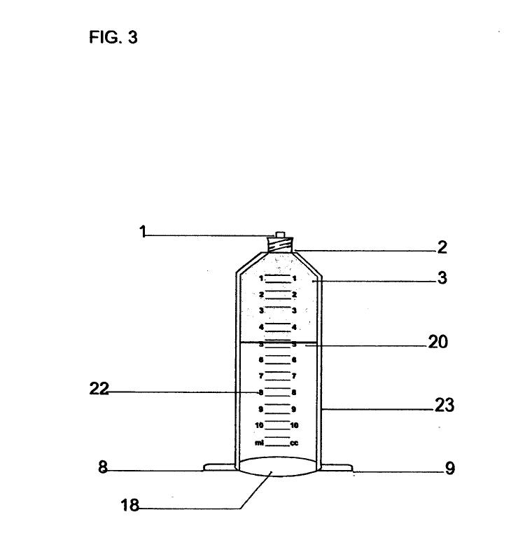

FIG. 3 is an exploded perspective view, showing the Housing barrel for FIG. 2. 1 is a circular tip with a small opening to allow an open free flow. 2 is a circular piece that is screwed onto other attachments (devices). 3 is an example of any fluids, body or etc. occupying the hollow space of the barrel. 19 is the bottom housing barrel where the collection takes place. 8 is the left finger bridge and 9 is the right finger bridge. 18 is the bottom opening of the housing barrel. 20 is the hollow inside of the housing barrel. 22 is the measuring scale to measure fluids. 23 is the housing barrel and is not limited to being produced with or without measuring scales on it and will accomplish the same goal.

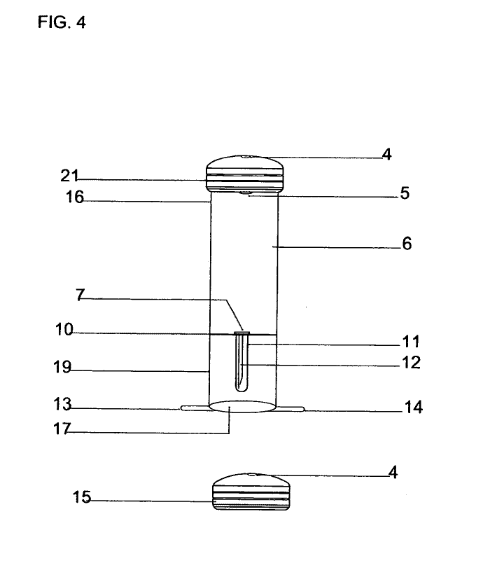

FIG. 4 is an exploded perspective view of FIG. 2, showing the second barrel separate from the housing barrel. Also showing the rubber attachment from the plastic attachment that have the exact shape and form. 4 is the small opening/hole on the top of the rubber stopper. 5 is the small opening/hole at the (16) and bottom (10) with small openings/holes. 10 is the base of the hollow barrel with small opening/hole. 7 is a small attachment feature. 11 is a rubber cover to protect the sharp object and free flow of fluids. 12 is a sharp object inside of 11 that allows fluids to flow free. 19 is a sectioned part of the second barrel where 11 & 12 reside for attachment container(s) for collecting fluids. 13 & 14 are finger bridges that allow control over the device and attachments. 17 is the bottom opening of the second barrel. bottom of the rubber stopper which is attached to 16 is the surface enclosed second barrel with a small opening/hole in the center of it. 15 is the rubber stopper with to small openings/hole in the top and bottom of it and not limited to other attachments or none at all to accomplish the same goal. 21 is the plastic attachment in the shape and form of the rubber stopper with a hole/opening at the top and bottom of it that is attached to 16 and is underneath 15. 6 is the second hollow barrel that have an enclosed top to it and bottom with the collection attached to it.

Although the proceeding description contains significant details, it should not be construed as limiting the scope of the invention but rather as providing illustrations of the preferred embodiments of the invention. As an example the rubber stopper illustrated could take many forms, one alternative as such variations would not materially alter the nature.

TABLE OF CONTENTS

| FORMS | |
|---|---|
| APPLICATION TRANSMITTAL | 1 |
| FEE TRANSMITTAL | 2 |
| MICRO ENTITY | 3 |
| APPLICATION DATA SHEET | 4 |
| SPECIFICATIONS | |
| TITLE OF INVENTION | 1 |
| CROSS REFERENCE | 2 |
| STATEMENT OF FEDERAL SPONSOR | 2 |
| MICROFICHE APPENDIX | 2 |
| BACKGROUND OF INVENTION | 3-10 |
| BRIEF SUMMARY OF BASIC FUNCTIONS | 11-12 |
| ASPECT OF THE DEVCE | 13 |
| BREIF DESCRIPTION VIEW OF DRAWINGS | 14-15 |
| REFERENCE NUMBERS TO DRAWINGS | 16-17 |
| DETAILED DESCRIPTION OF INVENTION | 18-22 |
| CLAIM | |
| CLAIM | 1-2 |
| ABSTRACT OF DISCLOSURE | 1 |
| TABLE OF CONTENT | 1 |

Having described my invention; I claim:

1. A Safety Syringe Collection Device configured for attachment to any line device for mode of transportation of fluids, the said line device using the SSCD Device;
Comprising:
A. A collection device configured to be attached to the said connective end of any line device.
B. The screw head of the SSCD Device is attached to said connective end of any line device by screwing on the housing barrel to connect.
C. The second barrel in pulled to abstract fluids into the said housing barrel;
D. Wherein the rubber stopper is attached to the top of the second barrel with a small opening/hole to allow free flow through it; but prevent leakage
E. Whereas the fluids travel through the rubber stopper into the hollow space of the second barrel filling with fluid(s);
F. The fluid(s) sit in the second barrel until a collection container(s) is connected to the bottom opening of the second barrel;
G. Once the connected container(s) is connected, the fluids travel from the second barrel into the attached feature;
H. After traveling through the attached feature, fluids flow through the sharp object into the collection container(s) once pushed on; the rubber covering will be pushed upward. It uncovers the sharp object by the rubber covering and activate free flow;
I. As stated in my claim, the finger bridge allows you to pull/push off container(s) with control and ease.
